(19) United States
(12) Patent Application Publication
(10) Pub. No.: US 2014/0264071 A1
(43) Pub. Date: Sep. 18, 2014

(54) Apparatus and Methods for Sanitizing Fluids in a Chamber

(71) Applicants: David A. Becker, Winona, MN (US); Sean W. Daley, Melrose, WI (US)

(72) Inventors: David A. Becker, Winona, MN (US); Sean W. Daley, Melrose, WI (US)

(73) Assignee: GEA FARM TECHNOLOGIES, INC., Naperville, IL (US)

(21) Appl. No.: 13/842,673

(22) Filed: Mar. 15, 2013

(51) Int. Cl. A61L 2/00

(52) U.S. Cl. CPC A61L 2/0047
USPC 250/435; 250/428

(57) ABSTRACT

A flow diverting lens through which sanitizing light is transmitted. The flow diverting lens alters fluid flow to enhance fluid exposure to the sanitizing light.

### BACKGROUND OF THE INVENTION

This invention relates generally to apparatus and methods for using light to sanitize fluids, and more particularly to flow diverting lenses for improving fluid exposure to the sanitizing light.

Bacteria growth in fluids is an ongoing problem. Particularly, milk in dairy harvesting and processing facilities is vulnerable to bacteria growth. The division of one bacterium into two daughter cells in a process called binary fission and results in an exponential growth of the bacteria over time. Bacteria growth can continue at a constant rate, so both the number of cells and the rate of population increase doubles with each consecutive time period. Spatially structured environments, such as biofilms or bacterial mats, present additional growth foundations. Biofilms, for example, can range from few micrometers in depth to half a meter in depth and contain multiple species. Bacteria protected within biofilms are much harder to kill then individual isolated bacteria and can have more than 500 times increased resistance to antibacterial agents than individual “planktonic” bacteria. Pathogenic bacteria are a major cause of animal death, disease, and infections in dairies.

Various methods for sanitizing fluids such as milk with light are known. See, for example, U.S. Pat. No. 8,127,667.

### SUMMARY OF THE INVENTION

The present invention is directed to a flow diverting lens for a light that has a wavelength sufficient to at least partially sanitize a fluid exposed to the light. The flow diverting lens can be of any desirable shape extending outwardly from a chamber wall or from any supporting structure to which it is joined so that it alters a flow path for fluid flowing past the lens. The shape can be a: dome; pyramid; crescent; ramp; or any shape that slows or diverts fluid flow to enhance light exposure, and therefore, treatment of the fluid. Causing the fluid to flow and altering flow paths can overcome additional problems caused by biofilms and bacterial mats, for example.

The flow diverting lens can divert fluid flow over, through or around the lens, or any combination thereof. It can be stationary along the wall of a chamber, movable either alone or with a mechanical mixing device, or in any location that improves exposure of the fluid to the sanitizing light. The flow of fluid can be laminar or turbulent, but by being diverted, its exposure to sanitizing light will be extended and improved.

The flow diverting lens can be formed integrally with a chamber wall, fixed surface, or a moveable mixing device. It can be open on a side that is opposite from the fluid flow to permit insertion of a light source and it can contain or otherwise encapsulate a light bulb, for example. The light source can be powered by battery, wired to a remote power source, or be powered by any suitable energy source.

In addition, the flow diverting lens can be part of a larger fluid flow diverter that may or may not be a lens and it may have proportions that are relatively opaque to the fluid treating light. For example, a flow diverter lens in accordance with the present invention can be dome-shaped and extend inwardly or outwardly from a chamber wall or other support surface. Adjacent to the flow diverter lens can be an embossment, debossment, ridge or any other shaped flow diverter, which may direct fluid flow toward the lens and the sanitizing light. The overall shape of the flow diverting lens and its adjacent flow diverter can act together to form a single flow diverter, or be separate flow diverters to cause flow in a desired direction or to create turbulence in the fluid for improved fluid exposure to sanitizing light.

The flow diverting lens preferably disperses sanitizing light, but it can also focus the sanitizing light to a desired point or area, such as a constrained flow path, or it can simply transmit light without substantially altering its focus of exposure.

The flow diverting lens can have a light source such as a UV light bulb disposed adjacent to or inside of it or at any suitable distance for a given application. Further, a single light source can be associated with any number of flow diverting lenses, but it is preferred to have one light bulb associated with one lens. Due to expense and energy requirements, it is preferred to use ultra-violet (“UV”) emitting LED light bulbs in conjunction with the flow diverting lens.

Preferably, the flow diverting lens extends outwardly from a chamber wall or other support surface and into the flow path of the fluid. Nonetheless, it is within the scope of the present invention to have a lens that extends away from the fluid flow path (such as a concave surface), so that fluid flow is diverted into and back out of the lens. In this optional way, the fluid can be treated and/or turbulated for improved exposure to sanitizing light.

As indicated above, the flow diverting lens can be associated with a chamber wall, a stationary surface or movable device. When a chamber is used, it can be any suitable shape for a given application and can be oriented so that fluid flows through the chamber due to gravity or by a motive force such as a pump. When on a moveable device, the moveable device can be a turbulator blade for immersion into a tank, for example.

Sanitizing light transmitted through the flow diverting lens can be emitted at a constant rate or can fluctuate based on any desired fluid parameter, such as: flow rate; type; consistency; viscosity; end use; temperature; or other property. The number, size, shape, and spacing of flow diverting lenses can be selected based on the fluid's: type; flow rate; end purpose; degree of contamination; or other parameter. Sensors can be used to detect the relative degree of contamination in the fluid both before and after treatment, and sensor data can be used to control light intensity, fluid flow rates, fluid temperature, recirculation, or other parameters affecting fluid sanitizing.

The sanitizing light can be activated (turned on) by any suitable means such as a switch. The switch can be turned on either manually or automatically, and the switch can be mounted on or otherwise activated by a device associated with the fluid flow diverting lens. For example, a pump used to cause fluid to flow past the flow diverting lens can be activated by a switch or controller that also activates (or turns on) the sanitizing light source. A valve or other moveable component can have a position sensor disposed on a moving part thereof, so that movement of that component can activate the light switch, either directly or indirectly.

Preferably, when multiple lenses are used, they are of the same shape and size to minimize manufacturing costs and for consistent sanitizing light emission. Nonetheless, the lenses can have any desired variety of shapes, sizes, and light transmission properties.

The present invention permits fluids to be sanitized at a variety of temperatures, including temperatures below what might be referred to as “pasteurizing” temperatures. Nonetheless, the flow diverting lenses in accordance with the present invention can be used in conjunction with more traditional pasteurizing processes or even in relatively cold temperatures associated with the dairy harvesting facility chillers and bulk storage tanks, for example.

### BRIEF DESCRIPTION OF THE DRAWINGS

FIG. 1 is a cross-sectional side view of a dairy fluid flow turbulator having flow diverting lenses, in accordance with the present invention;

FIG. 2 is a cross-sectional side view of a dairy fluid flow turbulator having flow diverting lenses, in accordance with the present invention;

FIG. 3 is a cross-sectional side view of a dairy fluid storage vessel having disposed therein a number of flow diverting lenses in accordance with the present invention;

FIG. 3A is an enlarged view of a flow diverting lens in FIG. 3;

FIG. 4 is a cross-sectional side view of a dairy fluid storage vessel having disposed therein a number of flow diverting lenses in accordance with the present invention;

FIG. 4A is an enlarged view of a flow diverting lens in FIG. 4;

FIG. 5 is a cross-sectional side view of a dairy fluid storage container with an agitation panel having flow diverting lenses disposed thereon, in accordance with the present invention;

FIG. 5A is an enlarged view of a flow diverting lens in FIG. 5;

FIG. 6 is a cross-sectional side view of a dairy fluid storage container with an agitation panel having flow diverting lenses disposed thereon, in accordance with the present invention;

FIG. 6A is an enlarged view of a flow diverting lens in FIG. 6;

FIG. 7 is a cross-sectional side view of a dairy fluid storage container with an agitation panel having flow diverting lenses disposed thereon, in accordance with the present invention;

FIG. 7A is an enlarged view of a flow diverting lens in FIG. 7;

FIG. 8 is a cross-sectional side view of a dairy fluid storage container with an agitation panel having flow diverting lenses disposed thereon, in accordance with the present invention;

FIG. 8A is an enlarged view of a flow diverting lens in FIG. 8;

FIG. 9 is a cross-sectional side view of a dairy fluid storage container with an agitation panel having flow diverting lenses disposed thereon, in accordance with the present invention; and

FIG. 9A is an enlarged view of a flow diverting lens in FIG. 9.

### DETAILED DESCRIPTION OF THE INVENTION

In the following detailed description of drawings, the same reference numeral will be used to identify the same or similar parts in each of the figures, unless otherwise noted.

Depicted in FIGS. 1 and 2 are cross-sections of a milk treatment turbulator 20 having a wall 21, an upstream end 22, a downstream end 24, and a bore 26 through which milk or other dairy fluid flows for treatment to kill or reduce bacteria in the dairy fluid. The dairy fluid is represented by flow lines 28.

The fluid flow lines 28 illustrate a generally straight flow path through the turbulator inlet 22, and then diverge in a number of directions as they contact flow diverting lenses 30, of which they are preferably a sufficient number to turbulate the dairy fluid for adequate exposure to sanitizing light, as explained below. The fluid flow lines 28 are provided as a visual aid to understanding the effect of the flow diverting lenses 30, but they are not intended to show exact flow patterns or flow patterns at all depths. By causing the fluid to flow past or to be turbulated by the flow diverting lens 30, biofilms as an additional location for bacterial growth can be minimized. Thus, fluid flow reduces bacterial growth locations and improves exposure to treatment light.

The flow diverting lenses 30 are shaped, sized, and spaced apart to optimize dairy fluid exposure to sanitizing light by causing fluids to change proximity to the flow diverting lenses 30 and increase exposure time, as compared to direct flow at constant proximities to the wall 21.

The sanitizing light is provided by a suitable light source such as a UV light bulb 34, only one of which is illustrated in FIGS. 1 and 2, but it is understood that light bulbs 34 are disposed in or behind all or a majority of the flow diverting lenses 30.

The flow diverting lenses 30 are preferably made of a material that provides optimum translucence for the sanitizing light while also being of practical expense to make and maintain. The number and spacing of flow diverting lenses 30 can be determined, at least in part, using the dairy fluid's 28 flow rate, viscosity, temperature, degree of bacterial contamination, and sanitizing light intensity.

The shape of the flow diverting lenses 30 is typically selected based on obtaining the optimum light transmission properties, desired flow characteristics, ease of manufacturing, cost, for example. The flow diverting lens 30 is selected to have light transmission properties for a light source having a wavelength sufficient to at least partially sanitize a fluid exposed to the light. The flow diverting lens 30 can be of any desirable shape extending outwardly from a chamber wall 21 or from any supporting structure to which it is joined so that it alters a flow path for fluid flowing past the lens 30. The shape can be a: dome (FIGS. 1, 2, 3, 3A, 5, 5A, 6, 6A, 8, and 8A); conical (FIGS. 2, 4, 7, 9, and 9A); pyramid; crescent; ramp; or any shape that slows or diverts fluid flow to enhance light exposure, and therefore, treatment of the fluid.

The flow diverting lens 30 can divert fluid flow over the lens, around the lens or both. It can be stationary along the wall of a chamber, or movable either alone or with a mechanical mixing device that improves exposure of the fluid to the sanitizing light. The flow of fluid can be laminar or turbulent, but by being diverted, its exposure to sanitizing light will be improved because more of the fluid will be brought into close exposure to the sanitizing light and exposure duration will be increased.

The flow diverting lens 30 can be formed integrally with a chamber wall, fixed surface, or a moveable mixing device. It can be open on a side that is opposite from the fluid flow to permit insertion of a light source and it can contain or otherwise encapsulate a light bulb, for example. The light source can be powered by battery, wired to a remote power source, or be powered by any suitable energy source.

In addition, the flow diverting lens 30 can be part of a larger fluid flow diverter that may or may not be a lens and the part that is not the lens can be relatively opaque to the fluid treating light. For example, a flow diverting lens 30 in accordance with the present invention can be dome-shaped and extend inwardly or outwardly from a chamber wall or other support surface. Adjacent to the flow diverting lens 30 can be an embossment, debossment, ridge or any other shaped flow diverter, which may direct fluid flow toward the lens. The overall shape of the flow diverting lens 30 and its adjacent flow diverter can act together to form a single flow diverter, or as separate flow diverters to cause flow in a desired direction or to create turbulence in the fluid for improved fluid exposure to sanitizing light.

The flow diverting lens 30 preferably disperses sanitizing light, but it can also focus the sanitizing light to a desired point or area, such as a constrained flow path, or it can simply transmit light without substantially altering its exposure.

The flow diverting lens 30 can have a light source such as a UV light bulb 34 disposed adjacent to or inside of it or at any suitable distance for a given application. Further, a single light source can be associated with any number of flow diverting lenses, but it is preferred to have one light bulb 34 associated with one lens 30. Due to their relatively low expense and energy requirements, it is preferred to use LED light bulbs in conjunction with the flow diverting lens.

Preferably, the flow diverting lens 30 extends outwardly from a chamber wall or other support surface and into the flow path of the fluid. Nonetheless, it is within the scope of the present invention to have a lens that extends away from the fluid flow path and has a concave surface, so that fluid flow is diverted into and back out of the lens 30. In this optional way, the fluid can be treated and/or turbulated for improved proximity and duration exposure to sanitizing light.

As indicated above, the flow diverting lens 30 can be associated with a chamber wall 21, a stationary surface or movable device. When a chamber is used, it can be any suitable shape for a given application and can be oriented so that fluid flows through the chamber 22 due to gravity or by a motive force such as a pump. When on a moveable device, the moveable device can be a turbulator blade 40 (FIG. 5), turbulator housing 42 (FIGS. 6 and 7), or a turbulator shaft 43 (FIGS. 8 and 9) for immersion into a tank 44, for example.

Sanitizing light transmitted through the flow diverting lens 30 can be emitted at a constant rate or can fluctuate based on any desired fluid parameter, such as: flow rate; type; consistency; viscosity; end use; temperature; or other property. The number, size, shape, and spacing of flow diverting lenses can be selected based on the fluid's: type; flow rate; end purpose; degree of contamination; or any other parameter. Sensors (not illustrated) can be used to detect the relative degree of contamination in the fluid before and after treatment, and sensor data can be used to control light intensity, fluid flow rates, fluid temperature or other parameters affecting fluid sanitizing.

The sanitizing light can be activated (turned on) by any suitable means such as a switch (not illustrated). The switch can be turned on either manually or automatically, and the switch can be mounted on or otherwise activated by a device associated with the fluid flow diverting lens 30. For example, a pump used to cause fluid to flow past the flow diverting lens can be activated by a switch or controller that also activates (or turns on) the sanitizing light source. A valve or other moveable component can have a position sensor disposed on a moving part thereof, so that movement of the part can activate the light switch, either directly or indirectly.

Preferably, when multiple lenses 30 are used, they are of the same shape and size to minimize manufacturing costs and for consistent sanitizing light emission. Nonetheless, the lenses can have any desired variety of shapes, sizes and light transmission properties.

The present invention permits fluids to be sanitized at a variety of temperatures, including temperatures below what might be referred to as pasteurizing temperatures. Nonetheless, the flow diverting lenses in accordance with the present invention can be used in conjunction with more traditional pasteurizing processes or even in relatively cold temperatures associated with the dairy harvesting facility chillers and bulk storage tanks, for example.

1. Apparatus for sanitizing a fluid with sanitizing light, the apparatus comprising:
a first chamber wall at least partially defining a fluid flow path; and
a flow diverting lens through which sanitizing light is transmitted, and the flow diverting lens is joined to the first chamber wall to at least partially alter the fluid flow path.

2. The apparatus of claim 1, wherein the first chamber wall is at least partially tubular in shape.

3. The apparatus of claim 1, wherein the first chamber wall is substantially flat.

4. The apparatus of claim 1, and further comprising:
a second chamber wall spaced apart from the first chamber wall to at least partially define the fluid flow path.

5. The apparatus of claim 1, and further comprising:
a second chamber wall spaced apart from the first chamber wall to at least partially define the fluid flow path; and
a second flow diverting lens through which sanitizing light is transmitted, and the second flow diverting lens is joined to the second chamber wall to at least partially alter the fluid flow path.

6. The apparatus of claim 1, and further comprising:
a second chamber wall spaced apart from the first chamber wall to at least partially define the fluid flow path, wherein the second chamber wall is substantially transparent to sanitizing light.

7. The apparatus of claim 1, and further comprising:
a second chamber wall spaced apart from the first chamber wall to at least partially define the fluid flow path, wherein the second chamber wall is substantially transparent to sanitizing light, and further comprising:
a sanitizing light source positioned to emit sanitizing light through the second chamber wall.

8. The apparatus of claim 1, wherein the flow diverting lens extends outwardly from the first chamber wall and into a fluid flow path to direct fluid flow around the flow diverting lens.

9. The apparatus of claim 1, wherein the flow diverting lens defines a recess through which at least a portion of the fluid flows.

10. The apparatus of claim 1, and further comprising:
a second flow diverting lens joined to the first chamber wall to at least partially alter the fluid flow path.

11. The apparatus of claim 1, and further comprising:
a flow diverting ridge extending outwardly from the first chamber wall disposed adjacent to the flow diverting lens.
